(19) United States
(12) Patent Application Publication
(10) Pub. No.: US 2003/0091472 A1
(43) Pub. Date: May 15, 2003

(54) Method and device for determining an abnormal skin dryness

(76) Inventors: Alois Cremerius, Dusseldorf (DE); Rudolf Thissen, Wassenberg (DE); Reinhard Zick, Lingen (DE)

Correspondence Address: Liniak Berenato & White, Suite 240, 6550 Rock Spring Drive, Bethesda, MD, 20817, US

(21) Appl. No.: 10/257,681

(22) PCT Filed: Apr. 3, 2001
(86) PCT No.: PCT/EP01/03776

(30) Foreign Application Priority Data
Apr. 15, 2000 (DE) 10018790.0

(51) Int. Cl. G01N 15/06; G01N 33/00

(52) U.S. Cl. 422/68.1

(57) ABSTRACT

A method for the determination of abnormal skin dryness includes providing a reaction support (2), in particular of textile, which is impregnated with a 3.15- to 25-weight percent cobalt(II) chloride solution and then dried until the cobalt(II) chloride has a blue color. The reaction support (2) is applied onto one side of a planar base element (1) made of a flexible material. The reaction support (2) is covered with a protective material (3) that is impermeable to air and moisture. The protective material (3) is removed immediately before the determination of skin dryness. The reaction support (2) is placed onto a skin area and secured, and is removed from the skin again after a defined time interval, with a color shift of the reaction support to red indicating a normal skin dryness.

The present invention concerns a method for the determination of abnormal skin dryness, and in particular for the diagnosis of diabetic foot syndrome.

In the context of the treatment of diabetes, early detection of sequelae, for example diagnosis of “diabetic foot syndrome” (DFS) and of polyneuropathy (PNP), is of considerable importance. In the Federal Republic of Germany alone, there are approximately 5 million diabetics who are all threatened by diabetic foot, which represents the most common complication of the metabolic disturbance. Disruptions in specific nerve functions, in combination with circulatory disruptions, result in cutaneous changes, and the latter in turn lead to practically untreatable deep tissue defects with subsequent infections. The affected limbs often need to be amputated. In Germany alone, the number of amputations of the lower extremities caused by diabetic foot syndrome is approximately 28,000 to 30,000 annually. In recent years it has not been possible to reduce this figure despite a wide variety of efforts; quite to the contrary, the amputation rate is rising by 2% per year. Only a practical early detection method, with equally practical skin care instructions based thereon, can provide assistance here. The first diagnostically accessible early symptom of diabetic foot that is relatively easy to influence therapeutically is abnormal skin dryness, which makes the skin on the foot inelastic and thus easy to damage mechanically. Very minor trivial injuries, and inevitable colonization with ubiquitous pathogens, then constitute the beginning of diabetic foot syndrome. A variety of devices for sensing skin dryness have accordingly been developed for early diagnosis of diabetic foot. DE 198 33 440 A1, for example, discloses an apparatus which senses skin dryness by way of electrical resistance or electrical conductivity. These devices are associated with a corresponding outlay in terms of equipment.

It is therefore the object of the invention to describe a method with which it is easy to determine whether or not the moisture content of a skin area, in particular the soles of the feet, is (still) in the normal range.

This object is achieved, according to the present invention, by a method in which

a reaction support, in particular of textile, is impregnated with a 3.15- to 2-weight percent cobalt(II) chloride solution and then dried until the cobalt(II) chloride has a blue color;

the reaction support is applied onto one side of a planar base element made of a flexible material;

the reaction support is covered with a protective material that is impermeable to air and moisture;

the protective material is removed immediately before the determination of skin dryness; and

the reaction support is placed onto a skin area and secured, and is removed from the skin again after a defined time interval, a color shift of the reaction support to red indicating a normal skin dryness.

The underlying idea of the invention is thus to determine the liquid content of 10 the perspiration on a skin area, for example the sole of the foot, semiquantitatively in a reproducible, standardized test that has little susceptibility to error and is easy to handle, by applying onto the particular skin area a reaction support that comprises, with cobalt(II) chloride, an indicator that in the dry state has a blue color which, in the presence of a certain moisture content that corresponds to normal skin moisture, changes color to red. Based on the color exhibited by the reaction support after it has been in contact with the skin for a few minutes, it is thus easy to determine whether or not abnormal skin dryness is present.

This method for the indication of abnormal skin dryness can be performed by means of a corresponding indicator bandage which comprises a planar base element made of a flexible material that is equipped on one side with an adhesive material and retains the reaction support on that side. This indicator bandage is enclosed in a protective layer or protective envelope that is impermeable to air and moisture and is not removed until shortly before use in order to prevent premature reaction of the cobalt(II) chloride.

With regard to further advantageous embodiments of the invention, the reader is referred to the dependent claims and to the description below of an exemplary embodiment referring to the appended drawings, in which:

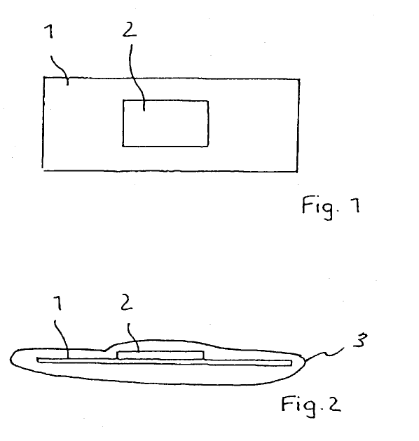

FIG. 1 shows an indicator bandage according to the present invention in a plan view; and

FIG. 2 shows the indicator bandage of FIG. 1 in a schematic side view.

The drawings depict an indicator bandage according to the present invention. The indicator bandage possesses a planar base element 1 made of a flexible material, in this case a transparent adhesive tape equipped with an adhesive layer on one side. Other usual bandage materials can also be used. Applied on the skin-facing side of adhesive film 1 having the adhesive layer is a textile reaction support 2 made e.g. of cellulose or nonwoven fabric, which is impregnated with a 6.25-weight percent cobalt(II) chloride solution. During manufacture, the cobalt(II) chloride solution is applied onto reaction support 2 and dried, for example by being heated to approximately 40° C., until its color is blue; reaction support 2 is then adhesively bonded onto bandage strip 1. It is clearly evident from the drawing that reaction support 2 is smaller than adhesive film 1 and is applied approximately centeredly onto it, so that an adhesive edge is created around reaction support 2, as is known from conventional bandages.

The bandage is preferably enclosed in a protective envelope 3 made of a 15 material that is impermeable to moisture, such as Kraftpaper, 40 g+20 g PE, which prevents the cobalt(II) chloride from coming into contact with moisture.

For early detection of abnormal skin dryness, the bandage is removed from the protective envelope 3 and adhesively bonded onto a skin area to be tested. If the skin possesses normal moisture, the blue color of the cobalt(II) chloride changes to a red color as a result of contact with skin perspiration. If insufficient skin moisture—in other words, abnormal skin dryness—exists, the blue color remains. In this fashion it is possible, with simple means, to ascertain reliably and semiquantitatively whether or not abnormal skin dryness is present. Tests have shown that with healthy skin, a color shift from blue through white and pink to red takes place within four minutes, whereas a pathological skin secretion definitely exists if such a color shift has not taken place after 20 minutes.

The method according to the present invention is suitable for diagnosis in all areas in which pathologically decreased sweat secretion plays a part. In the neurological field, in particular, it can be used instead of the Minor or Ninhydrin tests for detection of pathological sweat secretion.

1. A method for the determination of abnormal skin dryness, in which
a reaction support (2), in particular of textile, is impregnated with a 3.15- to 25-weight percent cobalt(II) chloride solution and then dried until the cobalt(II) chloride has a blue color;
the reaction support (2) is applied onto one side of a planar base element (1) made of a flexible material;
the reaction support (2) is covered with a protective material (3) that is impermeable to air and moisture;
the protective material (3) is removed immediately before the determination of skin dryness; and
the reaction support (2) is placed onto a skin area and secured, and is removed from the skin again after a defined time interval, a color shift of the reaction support to red indicating a normal skin dryness.

2. The method as defined in claim 1, wherein the reaction support (2) is impregnated with a 6.25-weight percent cobalt(II) chloride solution.

3. The method as defined in claim 1 or 2, wherein the reaction support (2) is made of cellulose.

4. The method as defined in claim 1 or 2, wherein a reaction support (2) made of a nonwoven material is used.

5. The method as defined in one of the foregoing claims, wherein the reaction support (2) is covered with a protective film (3) that is impermeable to air and moisture.

6. The method as defined in one of the foregoing claims, wherein a film that is adhesive on one side is used as the base element (1).

7. The method as defined in claim 6, wherein the reaction support (2) is smaller than the base element (1) and is applied, in particular, centeredly thereon.

8. An apparatus for the indication of abnormal skin dryness, having a planar base element (1) made of a flexible material that is equipped on its one side with an adhesive material and carries on that side a reaction support (2), in particular of textile, that is impregnated with a dried 3.15- to 25-weight percent cobalt(II) chloride solution and is covered with a protective layer (3) that is impermeable to air and moisture.

9. The apparatus as defined in claim 8, wherein the base element (1) is embodied as a film that is impermeable to air and moisture.

10. The apparatus as defined in claim 8 or 9, wherein the reaction support (2) is impregnated with a 6.25-weight percent cobalt(II) chloride solution.

11. The apparatus as defined in one of claims 8 through 10, wherein the reaction support (2) is made of a nonwoven material.

12. The apparatus as defined in one of claims 8 through 10, wherein the reaction support (2) is made of a cellulose material.
